SEQUENCE LISTING (3 sequences)

SEQ ID NO: 1 - DNA, 27 bp, Artificial sequence
  Synthetic antisense oligonucleotide
cagcaggagtggacagaagggctccat

SEQ ID NO: 2 - DNA, 26 bp, Artificial sequence
  Synthetic antisense oligonucleotide
agcggctgggatgcaccagggtccgg

SEQ ID NO: 3 - DNA, 25 bp, Artificial sequence
  Synthetic antisense oligonucleotide
tttcttgtcgttgctgctgaccacg

(19) United States
(12) Patent Application Publication
(10) Pub. No.: US 2003/0225026 A1
(43) Pub. Date: Dec. 4, 2003

(54) Methods, compositions and articles of manufacture useful for treating mammary tumors

(76) Inventors: Ari Elson, Rehovot (IL); Philip Leder, Chestnut Hill, MA (US)

Correspondence Address: G.E. EHRLICH (1995) LTD., c/o ANTHONY CASTORINA, SUITE 207, 2001 JEFFERSON DAVIS HIGHWAY, ARLINGTON, VA, 22202, US

(21) Appl. No.: 10/435,634

(22) Filed: May 12, 2003

Related U.S. Application Data
(60) Provisional application No. 60/379,385, filed on May 13, 2002.

(51) Int. Cl. A61K 48/00; C12Q 1/68; G01N 33/574; A61K 38/48; A61K 39/395

(52) U.S. Cl. 514/44; 424/94.63; 424/155.1; 435/6; 435/7.23

(57) ABSTRACT

A method of treating mammary tumors in a subject is provided. The method is effected by at least partially inhibiting receptor-type tyrosine phosphatase epsilon (RPTPe) activity or expression in mammary tumor tissue of the subject.

This application claims the benefit of priority from U.S. Provisional Patent Application No. 60/379,385, filed May 13, 2002.

### FIELD AND BACKGROUND OF THE INVENTION

The present invention relates to methods, compositions and articles of manufacture useful for treating mammary tumors and, more particularly, to methods, compositions and articles of manufacture designed for downregulating or inactivating receptor-type tyrosine phosphatase epsilon (RPTPe) activity or expression. The present invention further relates to method of reducing morphologic transformation and proliferation rate in a cell, cell culture or tissue by reducing RPTPe activity therein. The present invention further relates to a method of identifying an agent capable of inhibiting RPTPe activity, which can be tested for efficacy in breast cancer treatment.

Breast cancer is the most common type of cancer among American women with one in nine women developing the disease in her lifetime. Approximately 160,000 new cases of breast cancer are diagnosed each year. Most breast cancer cases occur in women over the age of 50 years. With earlier detection methods, breast cancer is now diagnosed at an early stage of development in the majority of women. However, despite the prevalence of early detection, survival rates at 5 years post diagnosis are only 50%. There are many treatments for breast cancer including surgery, radiation therapy, chemotherapy and hormonal therapy.

Breast surgery is generally traumatic for patients because it affects their self-image and libido. In addition, surgical procedures do not address metastatic foci that may be present in other parts of the body.

A wide variety of chemotherapy agents are employed in treatment of breast cancer. These agents commonly cause various side effects including hair loss, appetite suppression, weight reduction, immune system impairment, depression and fatigue. Side effects result from the generally cytotoxic nature of the chemicals employed.

Radiation therapy unavoidably affects healthy tissue causing side effects; tiredness, or fatigue and burns are common side effects. Some patients also experience breast soreness, swelling and reddening of the skin. In some patients, permanent darkening of the breast skin, change in the sensitivity of the breast skin, thickening of the breast skin, enlargement of the pores in the skin of the breast, or change in breast size also occur.

There is thus a widely recognized need for, and it would be highly advantageous to have, methods, compositions and articles of manufacture useful in treating mammary tumors devoid of the above limitations. Similarly, there is a great unmet need for a method of identifying a drug candidate useful in those methods, compositions and articles of manufacture.

### SUMMARY OF THE INVENTION

According to one aspect of the present invention there is provided a method of treating mammary tumors in a subject. The method includes at least partially inhibiting receptor-type tyrosine phosphatase epsilon (RPTPe) activity or expression in mammary tumor tissue of the subject.

According to another aspect of the present invention there is provided a pharmaceutical composition for treating mammary tumors. The composition includes, as an active ingredient, a therapeutically effective amount of an agent capable of at least partially inhibiting RPTPe activity or expression and a physiologically acceptable carrier and/or excipient.

According to yet another aspect of the present invention there is provided an article of manufacture including packaging material and a pharmaceutical composition identified for treatment of mammary tumors being contained within the packaging material. The pharmaceutical composition includes, as an active ingredient, an agent capable of at least partially inhibiting RPTPe activity or expression and a pharmaceutically acceptable carrier.

According to still another aspect of the present invention there is provided a method of reducing morphologic transformation and proliferation rate in a cell, cell culture or tissue. The method includes at least partially inhibiting RPTPe activity or expression in the cell, cell culture or tissue.

According to an additional aspect of the present invention there is provided a method of identifying a drug candidate for treatment of mammary tumors. The method includes screening a plurality of molecules for a molecule capable of at least partially inhibiting RPTPe activity or expression The molecule capable of inhibiting RPTPe activity or expression becomes the drug candidate.

According to still further features in the described preferred embodiments the at least partially inhibiting is accomplished by gene knockout.

According to still further features in the described preferred embodiments the introducing is effected via systemic administration of the agent.

According to still further features in the described preferred embodiments the subject is a human being.

According to still further features in the described preferred embodiments the agent capable of at least partially inhibiting RPTPe is a phosphatase inhibitor.

According to further features in preferred embodiments of the invention described below, at least partially inhibiting is effected by introducing into the mammary tumor tissue an agent selected from the group consisting of: (a) a molecule which binds RPTPe; (b) an enzyme which cleaves RPTPe; (c) an antisense polynucleotide capable of specifically hybridizing with an mRNA transcript encoding RPTPe; (d) a ribozyme which specifically cleaves RPTPe transcripts; (e) a non-functional analogue of at least a catalytic or binding portion of RPTPe; (f) a molecule which prevents RPTPe activation or substrate binding; (g) an siRNA molecule capable of inducing degradation of RPTPe transcripts; and (h) a DNAzyme which specifically cleaves RPTPe transcripts or DNA.

According to additional further features in the described preferred embodiments at least partially inhibiting is effected by changing an activation state of RPTPe.

According to still further features in the described preferred embodiments the antisense polynucleotide includes a sequence selected from the group consisting of SEQ ID NOs: 1-3.

According to still further features in the described preferred embodiments the non-functional analogue is capable of binding an RPTPe binding site of Src.

According to still further features in the described preferred embodiments the non-functional analogue is a substrate-trapping mutant of RPTPe.

According to still further features in the described preferred embodiments the at least partially inhibiting is accomplished by genetic manipulation of the cell, cell culture or tissue.

According to still further features in the described preferred embodiments the screening is accomplished by measuring at least one parameter selected from the group consisting of RPTPe binding, specific binding to an RPTPe transcript, RPTPe cleavage, and binding to an RPTPe binding site. Alternately, or additionally, screening may be accomplished by measuring direct or indirect inhibition of RPTPe protein or a level of activation thereof.

According to still further features in the described preferred embodiments the RPTPe binding site is a binding site on Src.

According to still further features in the described preferred embodiments the screening is effected by at least one method selected from the group consisting of an antibody based assay, an assay for competitive inhibition of RPTPe binding, an assay of inhibition of RPTPe activity, an assay of specific RPTPe binding, an assay of specific binding to at least a portion of an RPTPe transcript and an assay of RPTPe molecular weight. Alternately, or additionally, screening may be by an assay which measures RPTPe transcript size or transcript amount.

The present invention successfully addresses the shortcomings of the presently known configurations by providing methods, pharmaceutical compositions and articles of manufacture specifically useful in treatment of mammary tumors. The present invention further provides novel methodology which can be utilized for discovering new drug candidates useful in treatment of mammary tumors.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In case of conflict, the patent specification, including definitions, will control. In addition, the materials, methods, and examples are illustrative only and not intended to be limiting.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIGS. 1A-C illustrate Characteristics of EKO/Neu and Neu mammary tumor cells. FIG. 1A—Protein blot documenting expression levels of Neu, RPTPe, and RPTPa in cell lines derived from Neu-induced mammary tumors of wild-type (WT) and PTPe-deficient (KO) mice. RPTPe is either fully glycosylated (heavy band at ˜105 kDa) or non-glycosylated (light band at ˜85 kDa). The anti-PTPe antibody used cross-reacts with RPTPa. FIG. 1B—Typical morphology of WT (line 1908) and RPTPe-deficient (line 7381) tumor cells grown in tissue culture. Phase light microscopy, original magnification X200. FIG. 1C—Altered growth properties of cell lines derived from Neu-induced mammary tumors in culture and in vivo, following injection into nude mice. Growth rate in culture is presented as the number of cells (mean35 SEM) present 4 days after passaging, relative to the number of cells present 1 day after passaging as described in the Examples section. Nude mice tumorigenesis results are presented as weight (mg±SEM) of excised tumor 15 days after injection of cells. *, p=0.0037; **, p<0.0001, by the Mann-Whitney test. Data are from 3 WT or heterozygous vs. 3 EKO cell lines, with 4-6 repeats for each cell line in each parameter shown.

FIGS. 2A-D illustrate reduced activity and altered phosphorylation of Src in mammary tumor cells lacking RPTPe. Tumor cells examined contain two (WT), one (Het) or no (KO) functional alleles of PTPe. FIG. 2A—Reduced Src kinase activity in mammary tumor cells lacking RPTPe. Bar diagram depicts relative activities (mean±SEM) of Src from WT/Het or PTPe-deficient (KO) cell lines as measured by allowing immune-precipitated Src to phosphorylate exogenous enolase substrate. Each category contains data from three independent cell lines, each measured 3-4 times. *-p=0.0091 by Student's t-test. FIG. 2B—Representative Src activity assay from tumor cells. Top: ³²P-labeled enolase substrate. Bottom: Src protein present in immunoprecipitates used in same assay shown in the top panel of this figure. FIG. 2C—Altered Src phosphorylation in RPTPe-deficient tumor cells, as estimated from protein blots probed with phosphorylation state-sensitive anti Src antibodies. Bar diagram depicts average levels of phospho-Y416 Src and phospho-Y527 Src of KO cells relative to those of WT/Het cells. Data (mean±SEM) represents 3-4 cell lines in each category, 3-5 repeats for each cell line; **-p<0.0005 by Student's t-test. FIG. 2D—Representative protein blots showing levels of phospho-Y416 Src (top panel) and phospho-Y527 Src protein (bottom panel). Total levels of Src protein in same lysates are also shown.

FIGS. 3A-C illustrate that expression of RPTPe or cyt-PTPe increases Src activity and affects Src phosphorylation in a manner opposite to that of RPTPe deletion. SYF fibroblasts were transfected with c-Src and with either RPTPe (R) or cyt-PTPe(cyt). FIG. 3A—Bar diagram of Src kinase activity (mean±SEM) towards enolase in cell lines expressing Src and PTPe, relative to activity in cells expressing Src alone. Similar results were obtained by analyzing Src autophosphorylation in these experiments (not shown). * - p=0.048; **-p=0.020 by Student's t-test. FIG. 3B—Bar diagram depicting levels (mean±SEM) of phospho-Y416 Src and phospho-Y527 Src in SYF cells, relative to those measured in cells transfected with Src alone. *-p=0.014; **-p<0.0035 by Student's t-test. FIG. 3C—Representative protein blots depicting levels of phospho-Y416 Src and phospho-Y527 Src (top two panels), as well as expression levels of Src (third panel) and PTPe (fourth panel). n=2-5 for each bar in panels A, B.

FIGS. 4A-B illustrate that a substrate-trapping mutant of RPTPe binds Src. FIG. 4A—Src was immune-precipitated from SYF cells, which were transfected with Src and either wild-type (WT) or the substrate-trapping D302A mutant (DA) of RPTPe; precipitated material was blotted for presence of associated RPTPe (top panel) and for precipitated Src (bottom panel); Ab—control precipitation reaction performed in absence of primary anti-Src antibody. FIG. 4B—Expression of WT or D302A RPTPe in transfected cells.

FIGS. 5A-C illustrate that expression of Src in Neu-induced mammary tumor cells that lack RPTPe rescues their morphology and increases their growth rate. Mammary tumor cells of PTPe-deficient mice (line 7381) were infected with retroviral vectors containing empty vector (mock), c-Src (WT Src) or constitutively active Src (Y527F Src). Cells were analyzed following selection in Puromycin and 10-14 days passaging. FIG. 5A—Protein blot depicting relative expression levels of Src in the three cell types. FIG. 5B—Typical morphology of the three cell types. Phase light microscopy, original magnification X200. FIG. 5C—Growth of the three cell types in culture. Shown is cell number (mean±SEM) at days 2-4 after plating relative to 1 day after plating. n=6 for each point. *-p=0.0395; **-p=0.010 by Student's t-test.

FIGS. 6A-B illustrate that expression of RPTPe in PTPe-deficient mammary tumor cells rescues their altered morphology phenotype. Mammary tumor cells of PTPe-deficient mice (line 7381) were infected with retroviral vectors containing empty vector (mock; M) or RPTPe. FIG. 6A—Protein blot depicting expression of RPTPe (glycosylated (*) and unglycosylated (**)) in infected cells. FIG. 6B—Typical morphology of the infected cells. Phase light microscopy, original magnification X200.

FIGS. 7A-F illustrate reduced activity and altered phosphorylation of Yes and Fyn in mammary tumor cells lacking RPTPe. Tumor cells examined contain two (WT), one (Het) or no (KO) functional alleles of RPTPe. FIG. 7A—Reduced Yes kinase activity in mammary tumor cells lacking RPTPe. Bar diagram depicts relative activity (mean±SEM) of Yes from PTPe-deficient (KO) cell lines relative to that of WT/Het cells. Activities have been normalized to amount of Yes protein present in each assay. Similar results were obtained when Yes autophosphorylation was examined (not shown). Each category contains data from three independent cell lines, each measured 3-4 times. **-p=0.0029 by the Welch t-test test. FIG. 7B—Reduced amounts of Yes not phosphorylated at its inhibitory C-terminal Y535 in RPTPe-deficient tumor cells. Lysates of cells were immune-precipitated with the Src-2 antibody, which binds Src, Yes, or Fyn that are not phosphorylated at their C-terminal tyrosine (i.e. that are active). Precipitated material was then blotted with anti-Yes antibodies. Bar diagram depicts average levels of non-phospho-Y535 Yes in KO cells relative to those of WT/HET cells. Data (mean±SEM) represents 3 cell lines in each category, 3-4 repeats for each cell line; *-p=0.0118 by Student's t-test. FIG. 7C—Representative protein blots showing levels of non-phospho-Y535 Yes (top panel). Levels of total Yes in cell lysates (middle panel) and or RPTPe and RPTPa (bottom panel) are also shown. FIG. 7D—Reduced Fyn kinase activity in mammary tumor cells lacking RPTPe, similar to panel A. Each column summarizes data from three independent cell lines, each measured 4 times. *-p=0.025 by Welch's t-test. FIG. 7E—Reduced amounts of active Fyn not phosphorylated at its inhibitory C-terminal Y531 in RPTPe-deficient tumor cells, by immune-precipitation with the Src-2 antibody. Data (mean±SEM) represents 3 cell lines in each category, 5 repeats for each cell line; **-p=0.0027 by Student's t-test. FIG. 7F—Representative protein blots showing levels of non-phospho-Y531 Fyn (top panel). Levels of Fyn, RPTPe, and RPTPa are also shown. Note that Fyn phosphorylation is decreased in KO cell lines despite higher expression of Fyn proteins in these cells.

FIGS. 8A-D illustrate that expression of RPTPe or cyt-PTPe increases Yes specific activity and affects Yes phosphorylation in a manner opposite to that of RPTPe deletion. SYF fibroblasts were transfected with c-Yes and with either RPTPe (R) or cyt-PTPe(cyt). FIG. 8A—Bar diagram of Yes kinase activity towards enolase in cell lines expressing Yes and PTPe, relative to activity in cells expressing Yes alone. Activities have been normalized to amount of Yes protein present in each assay. Similar results were obtained by analyzing Yes autophosphorylation in these experiments (not shown). Data (mean±SEM) represents 3-7 repeats for each bar. **-p0.0016; *-p=0.025 by Welch's t-test. FIG. 8B—Representative protein blots depicting Yes activity assay. Top: ³²P-labeled enolase substrate. Middle: Yes protein present in immunoprecipitates used in the same assay shown in top panel. Bottom: PTPe expression levels in transfected SYF cells. FIG. 8C—Expression of RPTPe or cyt-PTPe decreases Yes phosphorylation at its negative regulatory site Y535. Bar diagram depicting average levels (mean±SEM) of Yes reactivity to the Src2 antibody relative to those measured in cells transfected with Yes alone. **-p=0.009; *-p=0.044 by Welch's t-test, n=3 for each bar. FIG. 8D±Representative protein blots showing levels of immunoprecipitated dephospho-Y535 Yes (top panel) as well as expression levels of Yes (middle panel) and PTPe (bottom panel) in the transfected cell lysates.

FIGS. 9A-D illustrate that expression of RPTPe or cyt-PTPe increases Fyn specific activity and affects Fyn phosphorylation in a manner opposite to that of RPTPe deletion. SYF fibroblasts were transfected with Fyn and with either RPTPe (R) or cyt-PTPe(cyt). FIG. 9A—Expression of RPTPe or cyt-PTPe increases Fyn activity. Bar diagram of normalized Fyn kinase activity (mean±SEM) towards exogenous enolase substrate in cells expressing Fyn and PTPe, relative to kinase activity in cells transfected with Fyn alone. Activities have been normalized to amount of Fyn protein present in each assay. Similar results were obtained by analyzing Fyn autophosphorylation in these experiments (data not shown). *-p=0.0421, **-p=0.0275 by Welch's t-test, n=7-9 for each bar. FIG. 9B—Representative protein blots depicting Fyn activity assay. Top: ³²P -labeled enolase substrate. Middle: Fyn protein present in immunoprecipitates used from the same assay as shown in top panel. Bottom: PTPe expression levels in transfected SYF cells. FIG. 9C—Expression of RPTPe or cyt-PTPe decreases Fyn phosphorylation at its inhibitory site Y531. Bar diagram depicting average levels (mean±SEM) of Fyn reactivity to the Src2 antibody relative to those measured in cells transfected with Fyn alone. **-p=0.0079, *-p=0.0²86 by the Mann-Whitney test, n=4-5 repeats for each bar. FIG. 9D—Representative protein blots showing levels of immunoprecipitated dephospho-Y531 Fyn (top panel) as well as expression levels of Fyn (middle panel) and PTPe (bottom panel) in the transfected cell lysates.

FIGS. 10A-B illustrate that RPTPe and Yes or Fyn are present in the same molecular complex. FIG. 10A—TOP: Yes was immunoprecipitated from SYF cells, which were transfected with Yes and either wild-type (WT) or the substrate-trapping D302A mutant (DA) of RPTPe. Precipitated material was blotted for the presence of associated RPTPe (top panel) and for precipitated Yes (bottom panel); Ab: negative control precipitation preformed in the absence of primary anti-Yes antibody. BOTTOM: Documentation of similar expression levels of WT and of D302A RPTPe in the transfected cell lysates. FIG. 10B—Similar analysis as in FIG. 10A, showing association between Fyn and WT or D302A RPTPe.

FIG. 11 illustrates that expression of Yes or Fyn in Neu-induced mammary tumor cells that lack RPTPe does not rescue their morphology. Mammary tumor cells of PTPe-deficient mice (line 7381) were infected with retroviral vectors containing empty vector (mock), c-Fyn, c-Yes, or c-Src. Cells were analyzed following selection in Puromycin and 10-14 days passaging. Typical morphology of the four transformed cell types described above is shown. Light microscopy, original magnification X200. Note morphological changes induced by Src are absent from cells expressing Fyn or Yes. Slight morphological changes were evident in cells expressing constitutively active Y535F Yes, while the active Y53 IF Fyn mutant had no effect (not shown).

### DESCRIPTION OF THE PREFERRED EMBODIMENTS

The present invention is of methods, compositions and articles of manufacture for downregulating or inactivating receptor-type tyrosine phosphatase epsilon (RPTPe) activity or expression which can be useful for treating mammary tumors. Specifically, the present invention can be used to reduce morphologic transformation and proliferation rate in a cell, cell culture or tissue by reducing RPTPe activity therein. The present invention further relates to a method of identifying an agent capable of inhibiting RPTPe activity, which agent can be developed into a drug suitable for treatment of breast cancer.

The principles and operation of methods, compositions and articles of manufacture according to the present invention may be better understood with reference to the drawings and accompanying descriptions.

Before explaining at least one embodiment of the invention in detail, it is to be understood that the invention is not limited in its application to the details of construction and the arrangement of the components set forth in the following description or illustrated in the drawings. The invention is capable of other embodiments or of being practiced or carried out in various ways. Also, it is to be understood that the phraseology and terminology employed herein is for the purpose of description and should not be regarded as limiting.

Members of the large protein tyrosine phosphatases (PTP) family of enzymes serve as key regulatory components in signal transduction pathways.

Specific members of the PTP family are the subject of numerous United States Patents (e.g. U.S. Pat. Nos. 5,693,488 to Fang et al.; 5,952,212 to Moller et al.; 5,821,084 to Olmsted et al; and 5,658,756; 5,866,397 and 6,214,564 to Rodan et al.), as well as numerous research publications, see for example, Krueger, N. X. et al. (1990) EMBO J. 9, 3241-3252; Liu, F. et al. (1998) Mol. Cell Biol. 18, 250-259; Peretz, A. et al. (2000) EMBO J. 19, 4036-4045 and Sato, T. et al. Science 268, 411-415 and references cited therein

Although several prior art studies have suggested a possible therapeutic role for some of these proteins [for a review, see, for example, Zhang, Z. Y. (2001) Curr Opin Chem Biol 5(4): 416-23 “Protein tyrosine phosphatases: prospects for therapeutics”], a specific method or composition useful in such therapy has not been published.

While reducing the present invention to practice, the present inventors have uncovered that RPTPe, a member of the PTP family of phosphatases plays a role in tumorogenesis of mammary tissue.

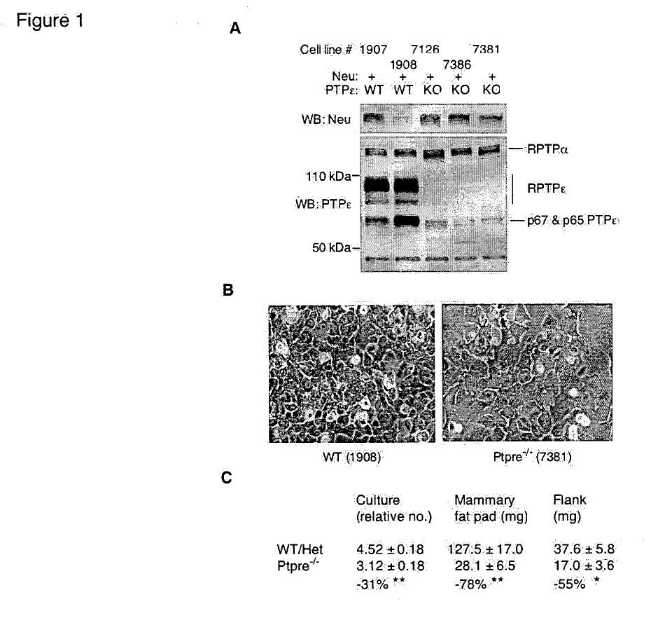

As is clearly illustrated in the Examples section which follows, breeding of mice expressing a Neu oncogene with mice in which the RPTPe gene has been “knocked out” produced mice with the same tumor latency as the Neu parental strain, but with an altered phenotype of mammary tumors (FIGS. 1A-C). In addition, cells isolated from mammary tumors which lacked a functional copy of the RPTPe gene exhibited a reduced growth rate, both in culture and when injected into nude mice (See Example 2 and FIGS. 1A-C). An alteration in cellular morphology was also observed in these cells. This demonstrates clearly that the RPTPe gene, and more specifically the RPTPe protein, play a central role in progression of mammary tumors.

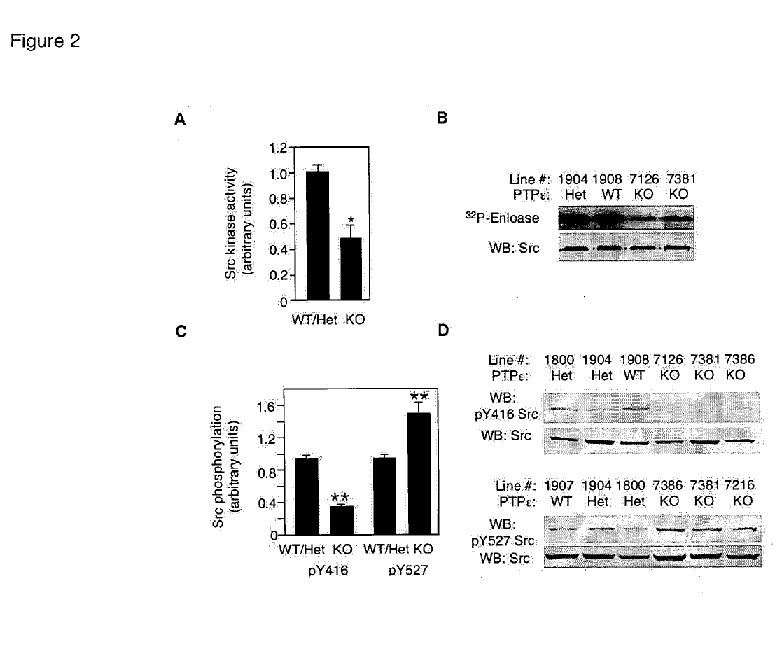

Further investigation, as detailed in examples 3, 4 and 5 (FIGS. 2A-6B) revealed that RPTPe exerts its effect on mammary tumor cells by altering the phosphorylation state of Src, a known oncogene. Most interesting from a clinical standpoint is the fact that while inhibition of RPTPe activity causes a change in tumor cell phenotype, subsequent restoration of normal RPTPe activity does not fully restore tumor malignancy.

Although the prior art documents cited above have described possible biological roles for several members of the PTP phosphatase family, none describe or suggest the role RPTPe plays in mammary tumors initiation and/or progression. Moreover, none of these prior art references contain a hint nor a suggestion that specifically inhibiting, or interfering with, RPTPe expression could provide the key to successful treatment or management of breast cancer.

According to one aspect of the present invention there is provided a method of treating mammary tumors in a subject, such as a male or female human. The method includes at least partially inhibiting receptor-type tyrosine phosphatase epsilon (RPTPe) activity or expression in mammary tumor tissue of the subject.

Several approaches for at least partially inhibiting receptor-type tyrosine phosphatase epsilon (RPTPe) activity or expression are envisaged by the present invention.

According to one preferred embodiment of this aspect of the present invention, partial or complete inhibition of RPTPe expression/activity can be accomplished by introducing into the subject an agent which is capable of partially or completely inhibiting RPTPe activity or an agent which is capable of partially or completely inhibiting expression.

Preferably, introducing is effected via systemic administration of the agent to a subject. Systemic administration may be effected by, for example, injection (e.g. intravenous, intramuscular, peritoneal or subcutaneous), oral administration, intraocular administration, intranasal administration, transdermal delivery, intravaginal administration or rectal administration. Further description of suitable routes of administration is provided herein below.

One example of an agent capable of partially or completely inhibiting RPTPe activity in the cell is a phosphatase inhibitor, such as for example, sodium pervanadate.

Additional agents suitable for inhibiting RPTPe activity in the cell include, but are not limited to, molecules which specifically bind RPTPE (e.g. antibody or an antibody fragment), enzymes which cleave RPTPe (e.g., calpain; Gil-Henn, H. et al. (2001) J. Biol. Chem. 276, 31772-31779), non-functional RPTPe analogues which are capable of blocking the Src binding site of RPTPe (e.g., the substrate-trapping mutant of RPTPe described in the Examples section which follows) or substrate analogues which are capable of competing for the RPTPe substrate binding or substrate catalytic region.

Complete or partial inhibition of RPTPe expression can be achieved using antisense oligonucleotides designed to specifically block transcription of RPTPe transcripts.

Design of antisense molecules which can be used to efficiently inhibit RPTPe expression must be effected while considering two aspects important to the antisense approach. The first aspect is delivery of the oligonucleotide into the cytoplasm of the appropriate cells, while the second aspect is design of an oligonucleotide which specifically binds the designated mRNA within cells in a way which inhibits translation thereof.

The prior art teaches of a number of delivery strategies which can be used to efficiently deliver oligonucleotides into a wide variety of cell types (see, for example, Luft (1998) J Mol Med 76(2): 75-6; Kronenwett et al. (1998) Blood 91(3): 852-62; Rajur et al. (1997) Bioconjug Chem 8(6): 935-40; Lavigne et al. (1997) Biochem Biophys Res Commun 237(3): 566-71 and Aoki et al. (1997) Biochem Biophys Res Commun 231(3): 540-5).

In addition, algorithms for identifying those sequences with the highest predicted binding affinity for their target mRNA based on a thermodynamic cycle that accounts for the energetics of structural alterations in both the target mRNA and the oligonucleotide are also available [see, for example, Walton et al. (1999) Biotechnol Bioeng 65(1): 1-9].

Such algorithms have been successfully used to implement an antisense approach in cells. For example, the algorithm developed by Walton et al. enabled scientists to successfully design antisense oligonucleotides for rabbit beta-globin (RBG) and mouse tumor necrosis factor-alpha (TNF alpha) transcripts. The same research group has more recently reported that the antisense activity of rationally selected oligonucleotides against three model target mRNAs (human lactate dehydrogenase A and B and rat gp130) in cell culture as evaluated by a kinetic PCR technique proved effective in almost all cases, including tests against three different targets in two cell types with phosphodiester and phosphorothioate oligonucleotide chemistries.

In addition, several approaches for designing and predicting efficiency of specific oligonucleotides using an in vitro system were also published (Matveeva et al. (1998) Nature Biotechnology 16, 1374-1375).

Several clinical trials have demonstrated safety, feasibility and activity of antisense oligonucleotides. For example, antisense oligonucleotides suitable for the treatment of cancer have been successfully used (Holmund et al. (1999) Curr Opin Mol Ther 1(3):372-85), while treatment of hematological malignancies via antisense oligonucleotides targeting c-myb gene, p53 and Bcl-2 had entered clinical trials and had been shown to be tolerated by patients [Gerwitz (1999) Curr Opin Mol Ther 1(3):297-306].

More recently, antisense-mediated suppression of human heparanase gene expression has been reported to inhibit pleural dissemination of human cancer cells in a mouse model [Uno et al. (2001) Cancer Res 61(21):7855-60].

Thus, the current consensus is that recent developments in the field of antisense technology which, as described above, have led to the generation of highly accurate antisense design algorithms and a wide variety of oligonucleotide delivery systems, enable an ordinarily skilled artisan to design and implement antisense approaches suitable for downregulating expression of known sequences without having to resort to undue trial and error experimentation.

Examples of antisense polynucleotide which can be used to specifically inhibit RPTPe expression in cells are set forth in SEQ ID NO 1-3.

These oligonucleotide sequences are provided as non-limiting examples of suitable antisense oligonucleotides for use in the context of the present invention. Each was demonstrated to be RPTPe specific by a BLAST DNA database search (J. Zhang et al. (1997) “PowerBLAST: A new network BLAST application for interactive or automated sequence analysis and annotation.” Genome Res. 7:649-656). Each of these oligonucleotides is specific to a region at, or immediately downstream of, the initiating ATG translation codon of the receptor-type form of PTPe. This region of the RPTPe transcript has been selected as being the most efficient place for interfering with translation according to antisense algorithms described hereinabove.

The antisense sequences described herein can also include a ribozyme sequence fused thereto. Such a ribozyme sequence can be readily synthesized using solid phase oligonucleotide synthesis.

RNA interference (RNAi) is yet another approach which can be utilized by the present invention to specifically inhibit RPTPe expression. RNA interference is a two step process. In the first step, which is termed as the initiation step, input dsRNA is digested into 21-23 nucleotide (nt) small interfering RNAs (siRNA), probably by the action of Dicer, a member of the RNase III family of dsRNA-specific ribonucleases, which processes (cleaves) dsRNA in an ATP-dependent manner. Successive cleavage events degrade the RNA to 19-21 bp duplexes (siRNA), each with 2-nucleotide 3′ overhangs [Hutvagner and Zamore (2002) Curr. Opin. Genetics and Development 12:225-232 and Bernstein (2001) Nature 409:363-366].

In the second step, termed the effector step, the siRNA duplexes bind to a nuclease complex to from the RNA-induced silencing complex (RISC). An ATP-dependent unwinding of the siRNA duplex is required for activation of the RISC. The active RISC then targets the homologous transcript by base pairing interactions and cleaves the mRNA into 12 nucleotide fragments from the 3′ terminus of the siRNA [Hutvagner and Zamore (2002) Curr. Opin. Genetics and Development 12:225-232, Hammond et al. (2001) Nat. Rev. Gen. 2:110-119, Sharp (2001) Genes. Dev. 15:485-90]. Although the mechanism of cleavage remains unresolved, research indicates that each RISC contains a single siRNA and an RNase [Hutvagner and Zamore (2002) Curr. Opin. Genetics and Development 12:225-232]. Because of the remarkable potency of RNAi, it has been suggested that the RNAi pathway employs an amplification step. Amplification could occur by copying of the input dsRNAs which would generate more siRNAs, or by replication of the siRNAs formed. Alternatively or additionally, amplification could be effected by multiple turnover events of the RISC [Hammond et al. (2001) Nat. Rev. Gen. 2:110-119, Sharp (2001) Genes. Dev. 15:485-90, Hutvagner and Zamore (2002) Curr. Opin. Genetics and Development 12:225-232]. For more information on RNAi see the following reviews Tuschl (2001) ChemBiochem. 2:239-245, Cullen (2002) Nat. Immunol. 3:597-599 and Brantl (2002) Biochem. Biophys. Act. 1575:15-25.

Synthesis of RNAi molecules suitable for use with the present invention can be effected as follows. First, the RPTPe mRNA sequence is scanned downstream of the AUG start codon for AA dinucleotide sequences. Occurrence of each AA and the 3′ adjacent 19 nucleotides is recorded as potential siRNA target sites. Preferably, siRNA target sites are selected from the open reading frame, as untranslated regions (UTRs) are richer in regulatory protein binding sites. UTR-binding proteins and/or translation initiation complexes may interfere with binding of the siRNA endonuclease complex [Tuschl ChemBiochem. 2:239-245]. It will be appreciated though, that siRNAs directed at untranslated regions may also be effective, as demonstrated for GAPDH wherein siRNA directed at the 5′ UTR mediated about a 90 % decrease in cellular GAPDH mRNA and completely abolished protein level (www.ambion.com/techlib/tn/91/912.html).

Following putative target site selection, target site sequences are compared to an appropriate genomic database (e.g., human, mouse, rat etc.) using any sequence alignment software, such as the BLAST software available from the NCBI server (www.ncbi.nlm.nih.gov/BLAST/). Putative target sites which exhibit significant homology to other coding sequences are filtered out.

Qualifying target sequences are selected as template for siRNA synthesis. Preferred sequences are those which include low G/C content, since such sequences have proven to be more effective in mediating gene silencing as compared to those having a G/C content higher than 55%. Several target sites are preferably selected along the length of the target gene for evaluation. For better evaluation of the selected siRNAs, a negative control is preferably used in conjunction. Negative control siRNA preferably include the same nucleotide composition as the siRNAs but lack significant homology to the genome. Thus, a scrambled nucleotide sequence of the siRNA is preferably used, provided it does not display any significant homology to any other gene.

Inhibition of RPTPe expression can also be effected using ribozymes. Ribozymes are being increasingly used for the sequence-specific inhibition of gene expression by the cleavage of mRNAs encoding proteins of interest [Welch et al., “Expression of ribozymes in gene transfer systems to modulate target RNA levels.” Curr Opin Biotechnol. October 1998; 9(5):486-96]. The possibility of designing ribozymes to cleave any specific target RNA has rendered them valuable tools in both basic research and therapeutic applications. In the therapeutics area, ribozymes have been exploited to target viral RNAs in infectious diseases, dominant oncogenes in cancers and specific somatic mutations in genetic disorders [Welch et al., “Ribozyme gene therapy for hepatitis C virus infection.” Clin Diagn Virol. Jul. 15, 1998; 10(2-3):163-71.]. Most notably, several ribozyme gene therapy protocols for HIV patients are already in Phase 1 trials. More recently, ribozymes have been used for transgenic animal research, gene target validation and pathway elucidation. Several ribozymes are in various stages of clinical trials. ANGIOZYME was the first chemically synthesized ribozyme to be studied in human clinical trials. ANGIOZYME specifically inhibits formation of the VEGF-r (Vascular Endothelial Growth Factor receptor), a key component in the angiogenesis pathway. Ribozyme Pharmaceuticals, Inc., as well as other firms have demonstrated the importance of anti-angiogenesis therapeutics in animal models. HEPTAZYME, a ribozyme designed to selectively destroy Hepatitis C Virus (HCV) RNA, was found effective in decreasing Hepatitis C viral RNA in cell culture assays (Ribozyme Pharmaceuticals, Incorporated—WEB home page).

DNAzymes can also be utilized by the present invention. DNAzymes are single-stranded polynucleotides which are capable of cleaving both single and double stranded target sequences (Breaker, R. R. and Joyce, G. Chemistry and Biology 1995; 2:655; Santoro, S. W. & Joyce, G. F. Proc. Natl, Acad. Sci. USA 1997; 943:4262) A general model (the “10-23” model) for the DNAzyme has been proposed. “10-23” DNAzymes have a catalytic domain of 15 deoxyribonucleotides, flanked by two substrate-recognition domains of seven to nine deoxyribonucleotides each. This type of DNAzyme can effectively cleave its substrate RNA at purine:pyrimidine junctions (Santoro, S. W. & Joyce, G. F. Proc. Natl, Acad. Sci. USA 199; for rev of DNAzymes see Khachigian, L M Curr Opin Mol Ther 2002; 4:119-21).

Examples of construction and amplification of synthetic, engineered DNAzymes recognizing single and double-stranded target cleavage sites have been disclosed in U.S. Pat. No. 6,326,174 to Joyce et al. DNAzymes of similar design directed against the human Urokinase receptor were recently observed to inhibit Urokinase receptor expression, and successfully inhibit colon cancer cell metastasis in vivo (Itoh et al., 20002, Abstract 409, Ann Meeting Am Soc Gen Ther www.asgt.org). In another application, DNAzymes complementary to bcr-abl oncogenes were successful in inhibiting the oncogenes expression in leukemia cells, and lessening relapse rates in autologous bone marrow transplant in cases of CML and ALL.

The agents described hereinabove can be administered to the subject per se or as part (active ingredient) of a pharmaceutical composition.

As used herein a “pharmaceutical composition” refers to a preparation of one or more of the active ingredients described herein with other chemical components such as physiologically suitable carriers and excipients. The purpose of a pharmaceutical composition is to facilitate administration of a compound to an organism.

Hereinafter, the phrases “physiologically acceptable carrier” and “pharmaceutically acceptable carrier” which may be interchangeably used refer to a carrier or a diluent that does not cause significant irritation to an organism and does not abrogate the biological activity and properties of the administered compound. An adjuvant is included under these phrases.

Herein the term “excipient” refers to an inert substance added to a pharmaceutical composition to further facilitate administration of an active ingredient. Examples, without limitation, of excipients include calcium carbonate, calcium phosphate, various sugars and types of starch, cellulose derivatives, gelatin, vegetable oils and polyethylene glycols.

Techniques for formulation and administration of drugs may be found in “Remington's Pharmaceutical Sciences,” Mack Publishing Co., Easton, Pa., latest edition, which is incorporated herein by reference.

Suitable routes of administration may, for example, include oral, rectal, transmucosal, especially transnasal, intestinal or parenteral delivery, including intramuscular, subcutaneous and intramedullary injections as well as intrathecal, direct intraventricular, intravenous, inrtaperitoneal, intranasal, or intraocular injections.

Alternately, one may administer the pharmaceutical composition in a local rather than systemic manner, for example, via injection of the pharmaceutical composition directly into a tissue region of a patient.

Pharmaceutical compositions of the present invention may be manufactured by processes well known in the art, e.g., by means of conventional mixing, dissolving, granulating, dragee-making, levigating, emulsifying, encapsulating, entrapping or lyophilizing processes.

Pharmaceutical compositions for use in accordance with the present invention thus may be formulated in conventional manner using one or more physiologically acceptable carriers comprising excipients and auxiliaries, which facilitate processing of the active ingredients into preparations which can be used pharmaceutically. Proper formulation is dependent upon the route of administration chosen.

For injection, the active ingredients of the pharmaceutical composition may be formulated in aqueous solutions, preferably in physiologically compatible buffers such as Hank's solution, Ringer's solution, or physiological salt buffer. For transmucosal administration, penetrants appropriate to the barrier to be permeated are used in the formulation. Such penetrants are generally known in the art.

For oral administration, the pharmaceutical composition can be formulated readily by combining the active compounds with pharmaceutically acceptable carriers well known in the art. Such carriers enable the pharmaceutical composition to be formulated as tablets, pills, dragees, capsules, liquids, gels, syrups, slurries, suspensions, and the like, for oral ingestion by a patient. Pharmacological preparations for oral use can be made using a solid excipient, optionally grinding the resulting mixture, and processing the mixture of granules, after adding suitable auxiliaries if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carbomethylcellulose; and/or physiologically acceptable polymers such as polyvinylpyrrolidone (PVP). If desired, disintegrating agents may be added, such as cross-linked polyvinyl pyrrolidone, agar, or alginic acid or a salt thereof such as sodium alginate.

Dragee cores are provided with suitable coatings. For this purpose, concentrated sugar solutions may be used which may optionally contain gum arabic, talc, polyvinyl pyrrolidone, carbopol gel, polyethylene glycol, titanium dioxide, lacquer solutions and suitable organic solvents or solvent mixtures. Dyestuffs or pigments may be added to the tablets or dragee coatings for identification or to characterize different combinations of active compound doses.

Pharmaceutical compositions which can be used orally, include push-fit capsules made of gelatin as well as soft, sealed capsules made of gelatin and a plasticizer, such as glycerol or sorbitol. The push-fit capsules may contain the active ingredients in admixture with filler such as lactose, binders such as starches, lubricants such as talc or magnesium stearate and, optionally, stabilizers. In soft capsules, the active ingredients may be dissolved or suspended in suitable liquids, such as fatty oils, liquid paraffin, or liquid polyethylene glycols. In addition, stabilizers may be added. All formulations for oral administration should be in dosages suitable for the chosen route of administration.

For buccal administration, the compositions may take the form of tablets or lozenges formulated in conventional manner.

For administration by nasal inhalation, the active ingredients for use according to the present invention are conveniently delivered in the form of an aerosol spray presentation from a pressurized pack or a nebulizer with the use of a suitable propellant, e.g., dichlorodifluoromethane, trichlorofluoromethane, dichloro-tetrafluoroethane or carbon dioxide. In the case of a pressurized aerosol, the dosage unit may be determined by providing a valve to deliver a metered amount. Capsules and cartridges of, e.g., gelatin for use in a dispenser may be formulated containing a powder mix of the compound and a suitable powder base such as lactose or starch.

The pharmaceutical composition described herein may be formulated for parenteral administration, e.g., by bolus injection or continuous infusion. Formulations for injection may be presented in unit dosage form, e.g., in ampoules or in multidose containers with optionally, an added preservative. The compositions may be suspensions, solutions or emulsions in oily or aqueous vehicles, and may contain formulatory agents such as suspending, stabilizing and/or dispersing agents.

Pharmaceutical compositions for parenteral administration include aqueous solutions of the active preparation in water-soluble form. Additionally, suspensions of the active ingredients may be prepared as appropriate oily or water based injection suspensions. Suitable lipophilic solvents or vehicles include fatty oils such as sesame oil, or synthetic fatty acids esters such as ethyl oleate, triglycerides or liposomes. Aqueous injection suspensions may contain substances, which increase the viscosity of the suspension, such as sodium carboxymethyl cellulose, sorbitol or dextran. Optionally, the suspension may also contain suitable stabilizers or agents which increase the solubility of the active ingredients to allow for the preparation of highly concentrated solutions.

Alternatively, the active ingredient may be in powder form for constitution with a suitable vehicle, e.g., sterile, pyrogen-free water based solution, before use.

The pharmaceutical composition of the present invention may also be formulated in rectal compositions such as suppositories or retention enemas, using, e.g., conventional suppository bases such as cocoa butter or other glycerides.

Pharmaceutical compositions suitable for use in context of the present invention include compositions wherein the active ingredients are contained in an amount effective to achieve the intended purpose. More specifically, a therapeutically effective amount means an amount of active ingredients (e.g. antisense oligonucleotide) effective to prevent, alleviate or ameliorate symptoms of a disorder (e.g., mammary tumor progression) or prolong the survival of the subject being treated.

Determination of a therapeutically effective amount is well within the capability of those skilled in the art, especially in light of the detailed disclosure provided herein.

For any preparation used in the methods of the invention, the therapeutically effective amount or dose can be estimated initially from in vitro and cell culture assays. For example, a dose can be formulated in an animal model, such as the murine Neu model (Muller et al., (1988) Cell 54, 105-115), to achieve a desired concentration or titer. Such information can be used to more accurately determine useful doses in humans.

Toxicity and therapeutic efficacy of the active ingredients described herein can be determined by standard pharmaceutical procedures in vitro, in cell cultures or experimental animals. The data obtained from these in vitro and cell culture assays and animal studies can be used in formulating a range of dosage for use in human. The dosage may vary depending upon the dosage form employed and the route of administration utilized. The exact formulation, route of administration and dosage can be chosen by the individual physician in view of the patient's condition. (See e.g., Fingl, et al., 1975, in “The Pharmacological Basis of Therapeutics”, Ch. 1 p. 1).

Dosage amount and interval may be adjusted individually to levels of the active ingredient are sufficient to retard tumor progression (minimal effective concentration, MEC). The MEC will vary for each preparation, but can be estimated from in vitro data. Dosages necessary to achieve the MEC will depend on individual characteristics and route of administration. Detection assays can be used to determine plasma concentrations.

Depending on the severity and responsiveness of the condition to be treated, dosing can be of a single or a plurality of administrations, with course of treatment lasting from several days to several weeks or diminution of the disease state is achieved.

The amount of a composition to be administered will, of course, be dependent on the subject being treated, the severity of the affliction, the manner of administration, the judgment of the prescribing physician, etc.

Compositions of the present invention may, if desired, be presented in a pack or dispenser device, such as an FDA approved kit, which may contain one or more unit dosage forms containing the active ingredient. The pack may, for example, comprise metal or plastic foil, such as a blister pack. The pack or dispenser device may be accompanied by instructions for administration. The pack or dispenser may also be accommodated by a notice associated with the container in a form prescribed by a governmental agency regulating the manufacture, use or sale of pharmaceuticals, which notice is reflective of approval by the agency of the form of the compositions or human or veterinary administration. Such notice, for example, may be of labeling approved by the U.S. Food and Drug Administration for prescription drugs or of an approved product insert. Compositions comprising a preparation of the invention formulated in a compatible pharmaceutical carrier may also be prepared, placed in an appropriate container, and labeled for treatment of an indicated condition, as if further detailed above.

Inhibition of receptor-type tyrosine phosphatase epsilon (RPTPe) expression can also be accomplished by gene knockout of the PTPe gene as is clearly illustrated by the results provided in the Examples section which follows.

As such, knock out constructs and strategies can be effectively used to inhibit PTPe expression. Such constructs can also be used in somatic and/or germ cells gene therapy to destroy activity of a PTPe allele to thereby dowiiregulate PTPe activity, as required. Further detail relating to the construction and use of knockout constructs is provided in the Examples section that follows and references cited therein. Additional detail can be found in Fukushige, S. and Ikeda, J. E.: Trapping of mammalian promoters by Cre-lox site-specific recombination. DNA Res 3 (1996) 73-80; Bedell, M. A., Jenkins, N. A. and Copeland, N. G.: Mouse models of human disease. Part I: Techniques and resources for genetic analysis in mice. Genes and Development 11 (1997) 1-11; Yoshimura I, Suzuki S and Hayakawa M.: Application of Cre-loxP system to the urinary tract and cancer gene therapy. Mol Urol. (2001) 5(2):81-4; Yu Y and Bradley A.: Engineering chromosomal rearrangements in mice. Nat Rev Genet. October 2001; 2(10):780-90.; Bermingham, J. J., Scherer, S. S., O'Connell, S., Arroyo, E., Kalla, K. A., Powell, F. L. and Rosenfeld, M. G.: Tst-1/Oct-6/SCIP regulates a unique step in peripheral myelination and is required for normal respiration. Genes Dev 10 (1996) 1751-62, which are incorporated herein by reference. Knocking-out of the PTPe gene in mice has already been accomplished (Peretz, A. et al. (2000) EMBO J. 19, 4036-4045).

Partial or complete inhibition of RPTPe activity or expression can also be used for reducing morphologic transformation and proliferation rate in a cell, cell culture or tissue.

Thus, according to another aspect of the present invention there is provided a method of reducing morphologic transformation and proliferation rate in a cell, cell culture or tissue.

The method according to this aspect of the present invention is effected by at least partially inhibiting RPTPe activity or expression in the cell, cell culture or tissue as described hereinabove. Reduction of morphologic transformation and proliferation rate may, in some cases, be most expediently accomplished by genetic manipulation of the cell, cell culture or tissue as described hereinabove.

In order to facilitate practice of the methods described hereinabove, and/or production of pharmaceutical compositions and articles of manufacture as described hereinabove, the present invention further provides a method of identifying a drug candidate for treatment of mammary tumors.

The method of identifying a drug candidate includes screening a plurality of molecules for a molecule capable of at least partially inhibiting RPTPe activity or expression. The molecule capable of inhibiting RPTPe activity or expression becomes the drug candidate.

Screening may be accomplished, for example, by measuring at least one parameter such as RPTPe binding, specific binding to an RPTPe transcript, RPTPe cleavage, or binding to an RPTPe binding site. Preferably, the RPTPe binding site is a binding site on Src. Alternately, or additionally, screening may be accomplished by measuring PTPe activity in the presence of the drug candidate.

As such, screening may be effected by a method or methods including, but not limited to, an antibody based assay, an assay for competitive inhibition of RPTPe binding, an assay of inhibition of RPTPe activity, an assay of specific RPTPe binding, an assay of specific binding to at least a portion of an RPTPe transcript and an assay of RPTPe molecular weight.

The following section describes in detail methodology which can be used for identifying a peptide drug candidate suitable for treatment of mammary tumors.

A peptide homologous to the region including the Y527 site of a previously characterized Src protein (e.g. NM009271 (mouse); M17031 (mouse Src, neuronal variant, identical to NM009271 in Y527 region); BC011566 (human); NM031977, AF157016 or AF130457 (Rat); or J00844, V00402 (chicken)) is synthesized and covalently bound to a suitable substrate (e.g. agarose or sepharose beads). For purposes of this specification and the accompanying claims, the term “Y527 site” of Src refers to amino acid number 527 in chicken Src, which is a tyrosine (Y). It is common practice in the art to refer to this residue by the chicken numbering (for historical reasons), even when working in other animals. Because of the high level of homology among Src gene products from different species, an analogue of Y527 exists in other species, although it is not necessarily the 527th residue. For example, the analogous tyrosine of position 527 in chicken is 534 in mouse, 529 in human, and 525 in Xenopus. A complex mixture of peptides, for example a crude proteolysed cell extract, is incubated with the substrate beads bearing the target molecule. The substrate beads bearing the target molecule are then washed to remove molecules which bind with low affinity. High affinity binding molecules are then eluted and collected. These are then incubated with substrate beads to which an irrelevant target molecule (e.g. a beta globin derived peptide) has been bound. The supernatant, containing molecules which did not bind during this second incubation, is collected and purified.

As an example, purification might include gel filtration chromatography and SDS-PAGE of eluted fractions followed by blotting to a membrane (e.g. PVDF or nitrocellulose), incubation with the Y527 target peptide and immunodetection employing an anti-Y527 primary antibody.

Duplicate blots would be subjected to similar treatment using an irrelevant target molecule (e.g. a keyhole limpet hemocyanin derived peptide) instead of Y527 and a primary antibody against keyhole limpet hemocyanin

Molecules which gave a positive result on the first blot and a negative result on the second blot would become candidates for additional purification steps, for example, HPLC purification of peptides eluted from PAGE gels. Once purified to homogeneity, these peptides can be sequenced and either produced synthetically, produced using recombinant DNA technology or derived from natural sources (via, for example, proteolysis).

Breast Cancer Patients often suffer from a reduced quality of life as a result of treatment side effects. In addition, the incidence of post-surgical metastatic growth at a remote location is high. According to the present invention which teaches an irreversible change in tumor cell phenotype, primary tumors are managed instead of removed.

Thus, the present invention represents a novel treatment modality for breast cancer which overcomes some of the limitations associated with prior art treatment approaches. Molecular intervention as taught by the present invention causes no change in self image for the patient because it requires no surgery. This fact alone will serve to reduce anxiety levels in the patient population and may lead to greater participation in early detection programs and/or genetic screening programs.

Further, the most effective breast cancer treatments currently available involve long term use of chemotherapeutic agents (e.g. tamoxifen). The toxic effects of these drugs are likely to be cumulative. The molecular intervention taught by the present invention causes a permanent change in tumor cell phenotype. Therefore, the length of treatment is finite. This means that any side effects from an RPTPe inhibiting agent will be of short duration. Alternately, or additionally, inhibitors of PTPe according to the present invention may act additively or synergistically with other drugs or treatments, thereby increasing their effectiveness, reducing treatment length and sparing patients from undesirable toxic side effects.

Finally, current medical theory is that primary tumors actually suppress growth of metastatic tumors by suppressing angiogenesis [reviewed in Folkman J. (2001)“Angiogenesis-dependent diseases” Semin Oncol. 28(6):536-42]. This implies that the present invention, which teaches management of growth rate of a primary tumor, represents a major breakthrough in treatment of metastatic cancer.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

### EXAMPLES

Reference is now made to the following examples, which together with the above descriptions, illustrate the invention in a non limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Current Protocols in Immunology” Volumes I-III Coligan J. E., ed. (1994); Stites et al. (eds), “Basic and Clinical Immunology” (8th Edition), Appleton & Lange, Norwalk, Conn. (1994); Mishell and Shiigi (eds), “Selected Methods in Cellular Immunology”, W.H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, Calif. (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

### General Materials and Methods

Expression vectors: Previously described eukaryotic expression vectors based on the pcDNA3 plasmid (Invitrogen) containing the cDNAs for mouse RPTPe (nucleotide coordinates 324-2423 of GenBank Accession number U35368), mouse cyt-PTPe (nucleotide coordinates 44-1972 of GenBank Accession number U36758), chicken c-Src (nucleotide coordinates 112-1713 of GenBank Accession number V00402) and chicken Y527F Src (Y-to-F change at amino acid position 527 of chicken Src; Genbank accession number V00402) (Gil-Henn et al., (2000) Oncogene 19, 4375-4384), as were similar vectors containing the cDNAs for mouse Yes (nucleotide coordinates 603-2228 of GenBank Accession number X67677) or human Fyn (nucleotide coordinates 580-2193 of GenBank Accession number MN002037). The D302A RPTPe mutant (D-to-A change at amino acid position 302 of mouse RPTPe; Genbank accession number U35368.) was generated by site-directed mutagenesis and, following sequence verification, cloned into the pcDNA3 plasmid. For retroviral infection studies, cDNAs for c-Src, Y527F Src, c-Yes and c-Fyn were cloned into the pBABE vector (Morgenstern and Land, (1990) Nucleic Acids Res. 18, 3587-3596).

Antibodies: Primary antibodies used in this study included polyclonal anti-PTPe (Elson and Leder, (1995a) J. Biol. Chem. 270, 26116-26122), monoclonal anti-v-Src (Calbiochem, La-Jolla, Calif., USA), polyclonal anti-phospho Y416 Src and anti-phospho Y527 Src (Biosource International, Camarillo, Calif.), monoclonal anti-Yes (clone 1, Transduction Laboratories, Lexington, Ky.), polyclonal anti-Fyn (Santa Cruz Biotechnology, Santa Cruz, Calif., for immunoprecipitation), monoclonal anti-Fyn (clone 25, Transduction Laboratories, for protein blotting), monoclonal anti-ErbB2 (clone 42, Transduction Laboratories), and polyclonal Src2 antibodies (Santa Cruz Biotechnology), The Src2 antibody recognizes the non-C-terminally phosphorylated forms of Src, Yes, and Fyn (Somani et al (1997), J. Biol. Chem. 272, 21113-21119). Secondary antibodies used were horseradish peroxidase-labeled goat-anti-mouse and goat-anti-rabbit immunoglobins (Jackson Immunoresearch Laboratories, West Grove, Pa., USA).

Generation of EKO/Neu mice: Gene-targeted mice lacking PTPe (EKO mice; C57Bl/6Jx129 genetic background; Peretz et al., (2000) EMBO J. 19, 4036-4045) were mated with MMTV-Neu transgenic mice (NF and NK lines; FVB/N genetic background; Muller et al., (1988) Cell 54, 105-115). Progeny were genotyped by DNA blot (Southern blotting) of tail biopsy samples and mated among themselves to generate MMTV-Neu mice homozygous for the PTPe-null allele (EKO/Neu mice), as well as EKO or MMTV-Neu mice for control purposes. Care was taken to generate and to compare mice of the same mix of genetic backgrounds. Female mice of each genotype were allowed to mate and nurse pups at will to promote expression of the MMTV-Neu transgene; mice were examined visibly or by palpation twice weekly for the presence of tumors.

EKOINeu tumor cell lines: Tumors from female EKO/Neu mice were minced in Dulbecco's Modified Eagle's Medium (DMEM), supplemented with 10% fetal bovine serum (Hyclone, Logan, Utah, USA), 4 mM glutamine, 50 units/ml penicillin G, and 50 □g/ml streptomycin. Each cell culture originated in a tumor from a separate mouse; cells were established in culture without need for additional transformation. Cell growth was quantified by seeding 0.5-1×10⁵ cells in duplicates in six-well plates, using the crystal violet method (Kueng et al., (1989) Anal. Biochem. 182, 16-19). Cell growth was followed for four days, with results from days 2-4 normalized to results obtained 16 hours after seeding to correct for possible variations in cell number or plating. Experiments were repeated 3-4 times for each cell line.

Propagation of tumors in nude mice: Equal numbers (1.5×106) of EKO/Neu tumor cells were injected in 0.2 ml PBS into the left #4 abdominal mammary gland or subcutaneously into the right flank of anesthetized 6-8 week old CD1 nude female mice. Mice were sacrificed 15 or 21 days later, and tumors were excised and weighed. Each cell line was assayed two or three times at each site, using three mice each time.

SYF cells: The previously describe SYF cell line (Klinghoffer et al., (1999) EMBO J.18, 2459-2471) was grown in DMEM medium, supplemented with 10% fetal calf serum (Life Technologies, Inc, Rockville, Md., USA), 4 mM glutamine, 1 mM sodium pyruvate and antibiotics as above.

Transfection: Cells were transfected using Lipofectamine 2000 (Life Technologies, Inc Rockville, Md., USA) according to the manufacturer's instructions. EKO/Neu cells were infected with pBABE-based retroviral vectors for c-Src or Y527F Src; cells were selected in 2 □g/ml Puromycin for two days, and analyzed for Src expression, morphology, and growth rates two weeks following selection.

Immunoprecipitation, SDS PAGE and immunoblotting: Cells were lysed in buffer A (50 mM Tris-Cl, pH 7.5, 100 mM NaCl, 1% Nonidet P-40), supplemented with 0.5 mM sodium pervanadate and protease inhibitors (1 mM N-(□-aminoethyl) benzene-sulfonyl fluoride, 40 micromolar bestatin, 15 micromolar E64, 20 micromolar leupeptin, 15 micromolar pepstatin; Sigma, St. Louis, Mo., USA). Sodium pervanadate was replaced with 5 mM iodoacetic acid in substrate-trapping experiments. 5-20 micrograms of total protein were analyzed on 7% or 10% SDS polyacrylamide gels, followed by transfer to nitrocellulose membranes (Protran, Schleicher & Schuell, Keene, N.H., USA), and hybridization to antibodies (All primary and secondary antibodies as described hereinabove in the antibodies section). Detection of antibody hybridization signals was performed by the enhanced chemiluminescence (ECL) technique, using a reagent kit from Pierce (Rockford, Ill., USA).

Protein transfer was monitored routinely by noting transfer of pre-stained molecular size marker proteins of the proper size range and by staining the blotted membranes with Ponceau S (Sigma, St. Louis, Mo., USA).

For immunoprecipitations, 0.5-1 mg of total cell protein were incubated with gentle shaking at 4° C. with 10 □g of anti-Src, anti-Fyn, or anti-Yes antibodies, followed by addition of goat-anti-mouse intermediary antibody and protein A-Sepharose beads (Amersham Pharmacia Biotech) for 3-4 h, after which the beads were washed extensively three times in RIPA buffer (for activity assays) or with buffer A (for substrate-trapping experiments), electrophoresed, and blotted. Experiments were repeated two to five times, and representative blots are shown.

Kinase activity assays: Src, Yes or Fyn were immunoprecipitated from 1 mg of cell lysate using Protein A-Sepharose beads as described hereinabove. Following immunoprecipitation, beads were equilibrated briefly in kinase buffer (20 mM MOPS, pH 7.0, 5 mM MgCl₂ for Src, 100 mM Hepes, pH7.0, 5 mM MnCl₂ for Yes or Fyn). Each reaction was conducted in 25 ml kinase buffer, to which 1 microliter (=5 □Ci) of gamma-³²P-ATP (3000 Ci/mmole, 10 mCi/mi, Amersham Pharmacia Biotech) and 5 micrograms acid-denatured enolase (Sigma, St. Louis, Mo., USA) were added. Tubes were briefly mixed and incubated with occasional mixing at 30° C. for 10 or for 30 minutes. During this period kinase activity was linear with respect to time. Reactions were stopped by adding SDS-PAGE sample buffer and boiling. Samples were electrophoresed and blotted onto membranes as described above. Radioactivity present in Src, Yes, or Fyn, as well as in enolase was quantified using a phosphorimager (BAS 2500, Fuji, Japan); the same blots were then probed with anti-Src, anti-Fyn, or anti-Yes antibodies and scanned with a scanning densitometer to allow normalization of kinase activity to the amount of kinase actually present in the immune-precipitates. Experiments were repeated three to five times for each kinase.

### Example 1

Creation of a Murine Breast Cancer Model Deficient in PTPe Gene Expression

In order to examine the role of RPTPe directly in vivo, PTPe-deficient mice expressing activated Neu in their mammary epithelial cells were created. This was accomplished by crossing two existing lines of mice. One line was a gene-targeted knock-out lacking PTPe and as such, lacks all four forms of the PTPe protein currently known to exist [EKO mice; Peretz et al., (2000) EMBO J. 19, 4036-4045, as well as Gil-Henn, H., Volohonsky, G., and Elson, A. (2001). Regulation of RPTP alpha and PTP epsilon by calpain-mediated proteolytic cleavage. J. Biol. Chem. 276, 31772-31779)] and the second line was a transgenic line carrying an activated Neu transgene controlled by the Mouse Mammary Tumor Virus (MMTV) promoter/enhancer (Neu mice; Muller et al., (1988) Cell 54, 105-115). The resultant hybrid mice (EKO/Neu mice) were expected to develop mammary tumors because of their MMTV driven Neu expression. Neu mice served as a control for tumor cell phenotype and tumor progression. EKO mice served as a control for baseline level of tumor incidence.

Tumor latency (i.e. the age at which mice developed detectable tumors) was similar in both EKO/Neu and Neu female mice. In both lines half the mice developed tumors by the age of approximately 130 days. A separate analysis of tumor growth rates was conducted (see below). As expected, EKO mice not expressing the MMTV-Neu transgene had a tumor incidence of 0% at the same age. Results from EKO and Neu mice confirm previously published reports. Results from the EKO/Neu mice demonstrate that lack of PTPe does not significantly hamper the ability of Neu to induce mammary tumors. This fact is not surprising since the MMTV-Neu transgene is known to have great transforming capability in the mammary gland system (Dankort and Muller, (2000) Oncogene 19, 966-967). Furthermore, expression of RPTPe protein in untransformed mammary epithelial tissue is very low, hence at the initiation of the transformation process there is no significant difference between wild-type mice, in which the PTPe gene is intact but very weakly expressed, and in EKO mice, in which the PTPe gene is disrupted and hence inactive. Significant levels of expression of RPTPe in mammary tumors occurs only at a later stage; the EKO/Neu mice therefore serve as a model system for further examination of mammary tumor progression in the absence of RPTPe as described hereinbelow.

### Example 2

### RPTPe Deficiency Influences Growth Rate of Mammary Tumor Cells

In order to further examine the effects of lack of PTPe on tumor cells, cell lines from several independent EKO/Neu or Neu mammary tumors were generated. All cell lines expressed the Neu transgene. Endogenous RPTPe protein was expressed only in cells from mice carrying at least one functional allele of PTPe (FIG. 1A). Tumor cells expressing PTPe behaved similarly whether they carried one or two functional PTPe alleles (HET and WT respectively in FIGS. 1A-C). All EKO/Neu and Neu tumor cells expressed significant and similar amounts of catalytically active RPTPa (FIG. 1A), a PTP closely related to PTPe. This clearly demonstrates that the observed phenotype is specifically attributable to diminished RPTPe activity. Importantly, while both EKO/Neu and Neu cells were of epithelial morphology, EKO/Neu cells were larger and flatter, and proliferated significantly slower than Neu cells (FIG. 1C). No differences in cell survival or plating efficiencies were observed between EKO/Neu and Neu tumor cells indicating that slower proliferation rate of EKO/Neu cell cultures was due to slower growth of these cells and not to increased mortality. EKO/Neu cells also tended to form smaller colonies than Neu cells when grown in soft agar, but variability among lines prevented this phenomenon from reaching statistical significance (not shown).

Differences in growth rates noted above persisted in vivo, following injection of EKO/Neu or Neu tumor cells into the mammary fat pad or subcutaneously into the flank of nude mice (FIG. 1C). Results of tumorigenesis in Nude mice are presented as weight of excised tumor 15 days after injection of cells. For each cell line at cach injection site, n=6. Excised tumor weight data was chosen in lieu of measuring tumor size (e.g. with calipers or micrometer) in live mice in order to eliminate errors stemming from irregular shape of many tumors and the tendency of mouse mammary tumors to form hollow, necrotic centers, which lead to overestimation of tumor mass based on its dimensions. EKO/Neu and Neu tumor cells formed tumors in vivo in nude mice. Tumors in mammary glands of recipient nude mice were significantly larger than those in the limbs of recipient nude mice.

The hypothesis that RPTPe suppression could retard tumor progression was supported by the fact that tumors which arose from EKO/Neu cells were significantly smaller than those of Neu cells in both the mammary fat pad (−78%) and flank (−55%) (FIG. 1C). Similar results were obtained in separate experiments in which tumors were harvested 21 days after injection of cells. Tumors produced in nude mice lacked visible signs of necrosis and were well vascularized. This fact indicates that the observed reduction in growth rate of EKO/Neu tumors in vivo in nude mice was not attributable to differences in cell survival or angiogenesis.

In summary, these results demonstrate that RPTPe is required for normal progression of Neu-induced mammary tumor cells both in vitro and in vivo in nude mice. Absence of RPTPe caused tumor cells to develop more slowly in this model system which has predictive value for human subjects.

### Example 3

### RPTPe Deficiency alters Src Phosphorylation and Reduces Src Activity

In order to discover the molecular mechanism underlying the observed retardation of tumor progression in RPTPe deficient tumor cells, additional experiments were undertaken. The fact that lack of PTPe affected growth rate of tumor cells generated by activated Neu but did not prevent appearance of tumors suggested that PTPe affects a collaborator of Neu rather than Neu itself.

Because Src tyrosine kinase is a known collaborator of Neu in transformation of mouse mammary epithelial cells (Dankort and Muller, (2000) Oncogene 19, 966-967); Muthuswamy and Muller, (1995) Oncogene 11, 1801-1810) and because Src can be dephosphorylated and activated by the related RPTPa in vitro and in vivo (Su et al., (1999) Curr. Biol. 9, 505-511; Ponniah et al., (1999) Curr. Biol. 9, 535-538), Src seemed a likely candidate for mediator of the observed phenotype in tumor cells derived from EKO/Neu mice.

Immunoblots of cell extracts from the various EKO/Neu cell lines using phospho-specific antibodies revealed that Src phosphorylation at its C-terminal inhibitory site Y527 (numbering as in chicken Src), was increased by 51% in EKO/Neu cells, while autophosphorylation at Y416 was reduced by 63% (FIGS. 2C and 2D). Both of these phosphorylation changes are known to cause reduced Src kinase activity (reviewed in Abram and Courtneidge (2000), Exp. Cell Res. 254(1), 1-13).

As expected, direct measurements revealed a two-fold reduction in Src activity in lysates of EKO/Neu cells (FIGS. 2A and 2B).

In order to establish that lack of PTPe was the direct cause of altered Src phosphorylation and activity in EKO/Neu cells, the effect of expressing PTPe on Src in transfected cells was examined. This examination was conducted using SYF cells (Klinghoffer et al., (1999) EMBO J. 18, 2459-2471) which are genetically deficient in the Src, Yes, and Fyn kinases and which do not express PTPe.

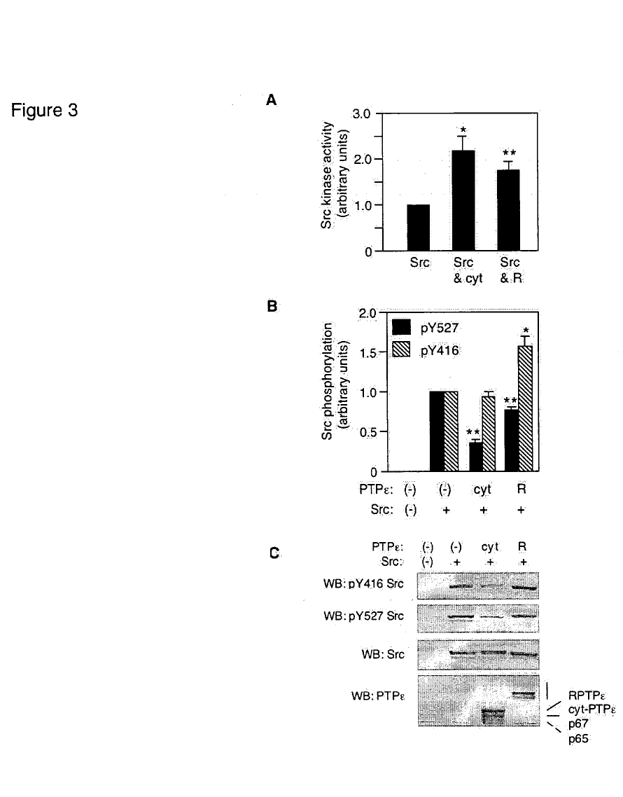

Co-expression of Src and of RPTPe in SYF cells resulted in changes in Src which were opposite from those observed in the PTPe-deficient EKO/Neu cells. In SYF cells expressing Src and RPTPe Y527 phosphorylation was decreased by 27% while Y416 phosphorylation was increased by 52%, and Src activity was increased by 78% (FIGS. 3A-C). Similar results were obtained in cells transfected with Src and RPTPa, confirming previously published studies (den Hertog et al., (1993) EMBO J. 12, 3789-3798; Harder et al., (1998) J. Biol. Chem. 273, 31890-31900; Zheng et al., (1992) Nature 359, 336-339; Zheng et al., (2000) EMBO. J. 19, 964-978).

These results are consistent with RPTPe preferentially dephosphorylating Src at Y527, thereby activating the kinase and resulting in increased autophosphorylation at Y416. Interestingly, the non receptor-type form of PTPe, cyt-PTPe, strongly reduced pY527 levels in transfected SYF cells by 66% (FIGS. 3B and 3C). Src activity was increased by 117% by cyt-PTPe (FIG. 3A), although no changes in pY416 levels were detected (FIGS. 3B and 3C). This is apparently due to stronger cyt-PTPe activity causing partial dephosphorylation at Y416 of Src, thereby countering autophosphorylation at this site. Similar levels of RPTPe and full-length cyt-PTPe were expressed in the SYF cells. The p67 PTPe and p65 PTPe, which are significantly co-expressed with cyt-PTPe, are exclusively cytosolic proteins and should not reduce phosphorylation of Src (Gil-Henn et al., (2000) Oncogene 19, 4375-4384; Gil-Henn et al., (2001) J. Biol. Chem. 276, 31772-31779).

### Example 4

### Src Interacts with the Active Site of PTPe

In order to conclusively demonstrate that Src is dephosphorylated directly by RPTPe, a substrate-trapping mutant of PTPe was employed. Mutants of this type, which are generated by mutating specific key residues in the catalytic domain of PTPs, are either virtually or entirely catalytically inactive but retain the ability to bind phosphorylated substrates via their catalytic site (Flint et al., (1997) Proc. Natl. Acad. Sci. U.S.A. 94, 1680-1685).

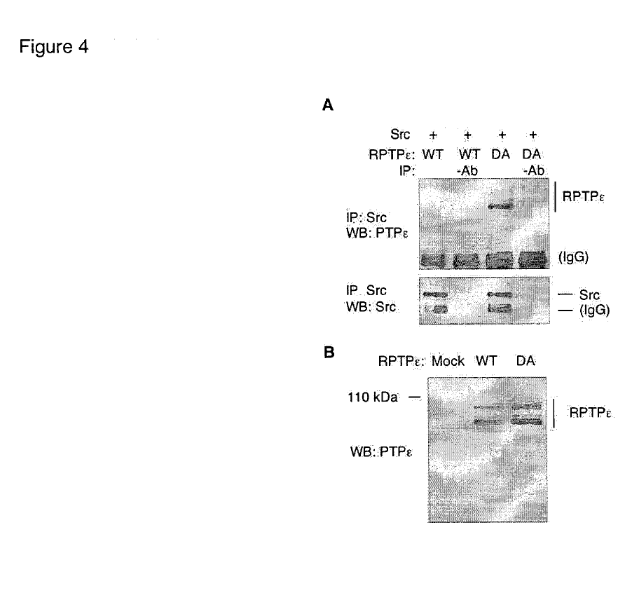

Src was co-expressed with wild-type RPTPe in SYF cells. Src was then immune-precipitated and blotted to reveal associated PTPe. Small amounts of WT RPTPe specifically associated with Src, and were not detected in identical experiments from which the primary precipitating anti-Src antibody was omitted (FIGS. 4A-B). Replacing WT RPTPe with the trapping mutant D302A RPTPe (Genbank accession U35368) resulted in significantly more RPTPe being co-precipitated with Src. Binding of D302A RPTPe was specific and was not detected in the absence of the precipitating antibody (FIGS. 4A-B). The increased binding of D302A RPTPe to Src indicates that Src interacts with the active site of RPTPe and demonstrates that Src is a substrate of RPTPe.

In summary, these results demonstrate that PTPe dephosphorylates and activates Src, and that lack of RPTPe is the cause of altered phosphorylation and reduced activity of Src in EKO/Neu cells. The relationship between Src and RPTPe provides a molecular mechanism for the observed retardation in growth rate of EKO/Neu tumor cells in culture and in vivo in nude mice.

### Example 5

### Src Expression Rescues Morphology and Growth Rate Phenotype of PTPe-Deficient Tumor Cells

In order to provide additional support for a PTPe-Src-phenotype connection supplementary expression of Src in EKO/Neu cells was undertaken. Two Src proteins were employed in an attempt to rescue some aspects of the phenotype of EKO/Neu tumor cells. EKO/Neu cells were infected with retroviral vectors expressing constitutively active (Y527F) Src or c-Src. Similar cells infected with empty vector served as controls in these experiments.

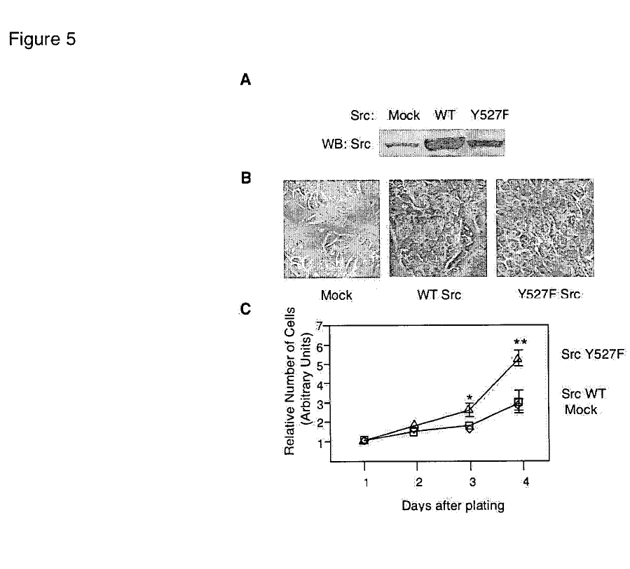

Y527F Src and c-Src and were detected in infected cells by protein blotting (FIG. 5A); expression of exogenous Y527F Src was lower than that of exogenous c-Src. It is believed that Y527F Src is harmful to cells so that only cells expressing low levels survive. Morphological examination of the infected cells (FIG. 5B) revealed that cells expressing either c-Src or Y527F-Src exhibited the same morphological characteristics found in Neu cells. Specifically, cells expressing c-Src or Y527F Src were smaller in size and exhibited denser growth, and a less-flattened morphology than EKO/Neu cells infected with an empty vector (FIG. 5B; mock).

Expression of Y527F Src in EKO/Neu cells also significantly increased the rate of cell proliferation as compared with cells expressing c-Src or infected with empty vector (FIG. 5C). Y527F Src appears to be more effective than c-Src in correcting the phenotype of EKO/Neu cells. This result is logical because Y527F Src is constitutively active and does not require activation by the (absent) RPTPe. These results confirm the role of Src in the phenotype observed in EKO/Neu tumor cells.

### Example 6

### Expression of RPTPe in EKO/Neu Cells Weakly Rescues the Observed Phenotype

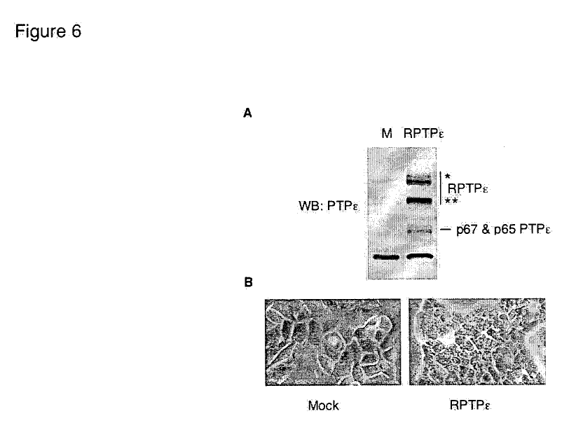

In an attempt to directly rescue the EKO/Neu cellular phenotype, a viral vector containing PTPe was employed. Following infection of EKO/Neu cells with a retroviral construct for RPTPe, Src activity was increased in cells expressing RPTPe by approximately 35%, and phospho-Y527 Src levels were reduced by 27% (not shown). As with cells expressing Src, EKO/Neu cells expressing RPTPe underwent morphological changes to resemble Neu cells or EKO/Neu cells expressing Src (FIG. 6B), although this morphological transition established slowly and the morphological changes observed were not as consistent as with cells expressing Src. The growth rate of the EKO/Neu cells expressing RPTPe remained consistent and was unchanged with respect to non-transformed cells.

In summary, the results presented herein clearly indicate that inhibition of RPTPe activity which reduces the total amount of phosphatase activity affecting Src results in reduced Src activity and retardation and possibly abolishment of tumor cell growth and proliferation.

### Example 7

### RPTPe Effect on Other Kinases

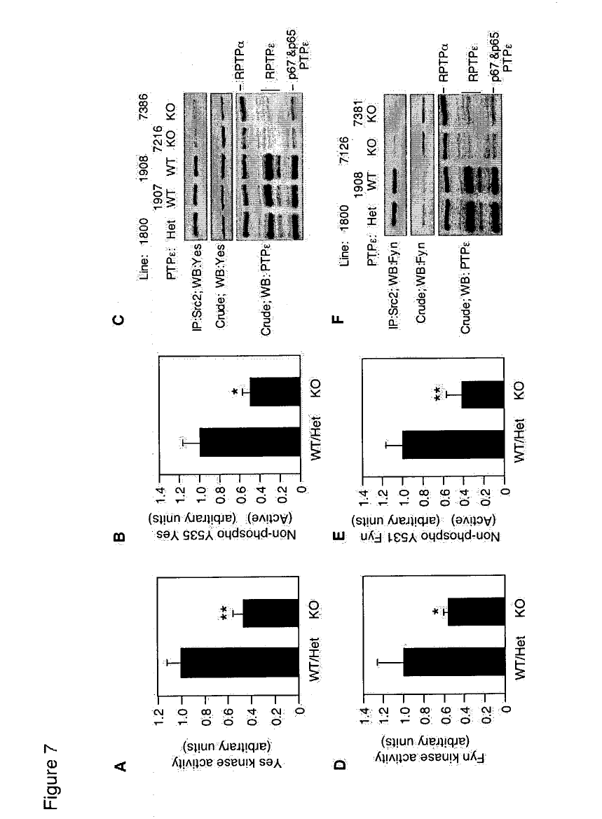

In order to determine whether lack of RPTPe could affect the specific activities of Fyn and Yes in mammary tumor cells, each kinase was immune-precipitated separately from tumor cells. in vitro measurements of kinase activity present in the precipitates revealed a decrease of approximately 50% in specific activities of either kinase in EKO/Neu cells (FIGS. 7A and 7D). In agreement, phosphorylation of the C-terminal tyrosine residue, which is a major negative regulatory site in Src-family kinases, increased in both kinases. In these studies, the kinases were immune-precipitated with an antibody that specifically recognizes Src-family kinase molecules that are not phosphorylated at this site (Y535 in mouse Yes, Y531 in mouse Fyn) and hence are active. Binding of this antibody to Fyn and Yes proteins expressed by EKO/Neu cells was reduced by 50-60%, indicating that phosphorylation of both kinases at this site was increased by similar proportions (FIGS. 7B, 7C, 7E, 7F). Of note, the specific activity and protein levels (FIG. 7F) of Fyn were similarly altered but in opposite directions in EKO/Neu cells, suggesting that total Fyn activity is under tight control. In this respect Fyn is distinct from Src and Yes, which exhibited unchanged protein levels but reduced specific activity.

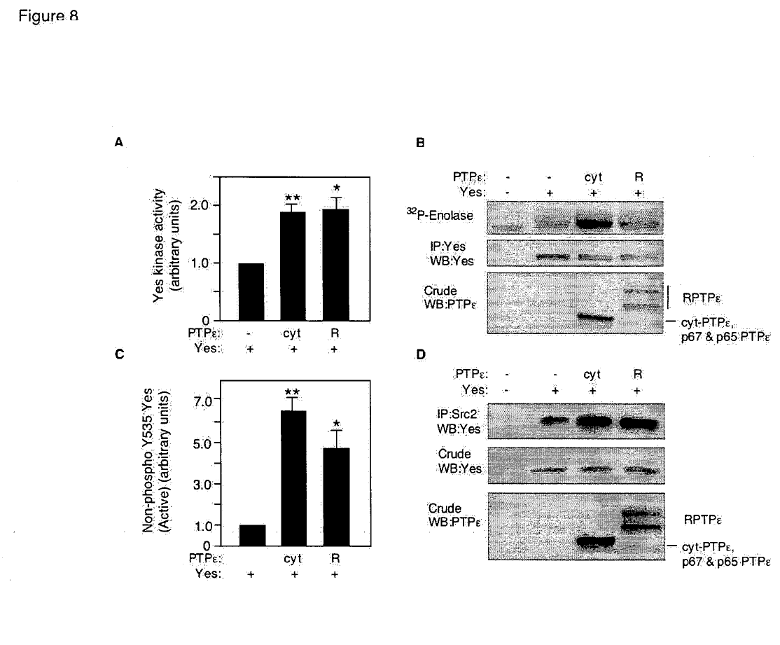

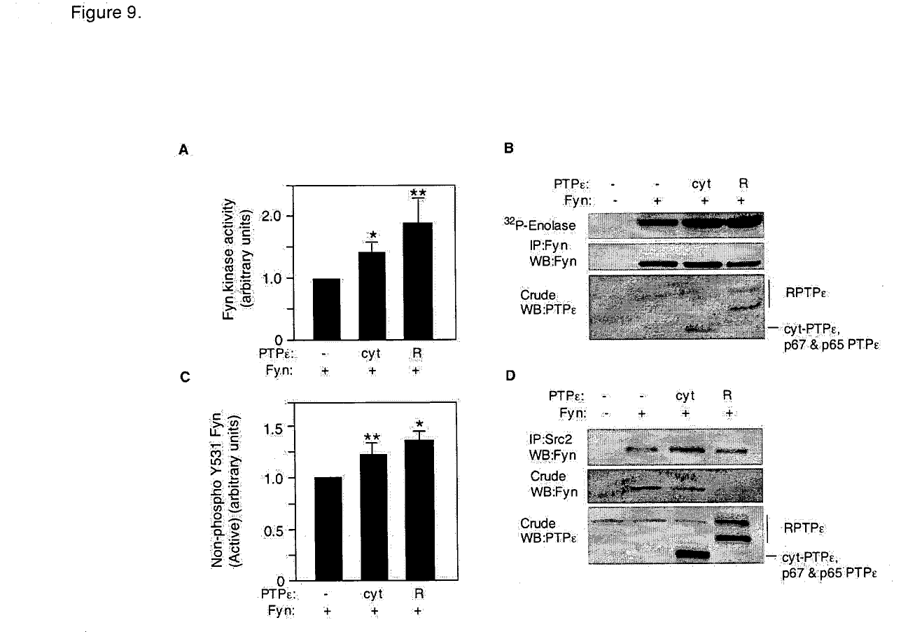

RPTPe activates Fyn and Yes: The above results indicated that lack of RPTPe correlates with decreased activity of Fyn and Yes. In order to determine whether lack of RPTPe merely correlates with or could be the cause of altered kinase phosphorylation and activity in EKO/Neu cells, the effect of expressing PTPE on both kinases in transfected cells was examined. In order to minimize background signals these studies were conducted in SYF cells, mouse embryo fibroblasts that are genetically deficient in Src, Yes, and Fyn [Klinghoffer et al., 1999 ibid], and do not express PTPe. Upon its expression in SYF cells, basal Yes kinase activity was detected, most likely the result of its dephosphorylation by other PTPs present in SYF cells. Co-expression of RPTPe nearly doubled the activity of Yes in these cells (FIG. 8A, 8B). Further studies revealed that expression of PTPe resulted in a 4.5- to 6.5-fold increase in binding of the dephospho-specific Src-2 antibodies (FIGS. 8C, 8D). This last result indicates that expression of PTPe causes a significant drop in Yes phosphorylation at its C-terminal inhibitory tyrosine, and is consistent with increased Yes activity noted in the same cells. Qualitatively similar effects were observed in experiments in which PTPe and Fyn were examined in the same system (FIGS. 9A-D). Expression of the non receptor-type form of PTPe, cyt-PTPe with either kinase produced results similar to those obtained with RPTPe (FIGS. 8A-D and 9A-D). A fraction of cyt-PTPe molecules are located at the cell membrane [Elson and Leder, 1995 ibid], and this form of PTPe can dephosphorylate some membrane-associated substrates, such as Src [Gil-Henn and Elson, 2003] and the voltage-gated potassium channels Kv1.5 and Kv2.1 [Peretz et al., 2000 ibid; Tiran et al. (2003) J. Biol. Chem. (in press)]. Expression of PTPe is then sufficient to reduce inhibitory phosphorylation of Yes and Fyn and to activate both kinases. Together with the opposite effects observed in mammary tumor cells lacking RPTPe, these results indicate that RPTPe is a physiological activator of Yes and of Fyn.

Yes and Fyn are present in molecular complexes with RPTPe: The ability of RPTPe to activate Yes and Fyn suggests that these kinases may be substrates of RPTPe. In order to address this issue the ability of a substrate-trapping mutant [Flint et al., 1997 ibid] of RPTPe to bind and precipitate each kinase was examined. Substrate-trapping mutants of PTPs are virtually or entirely inactive, but may retain the ability to interact with phosphotyrosine residues they would normally dephosphorylate via their catalytic site. In some cases, this association is strong enough to permit isolation of the substrate-enzyme complex [Flint et al., 1997 ibid].

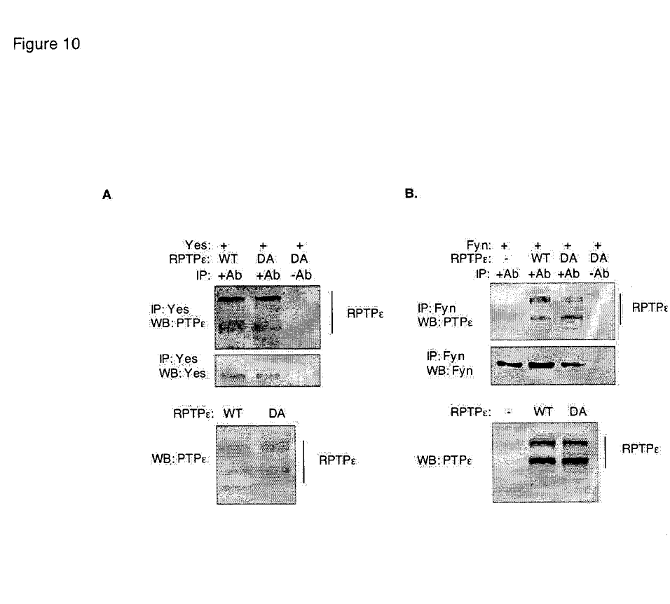

Following co-expression of Yes with wild-type RPTPe or with its D302A substrate trapping mutant in SYF cells, Yes was immune-precipitated and blotted to reveal associated RPTPe. These studies revealed significant association between Yes and RPTPe, both in its wild-type and D302A forms (FIG. 10A). Similar results were obtained in similar experiments using Fyn (FIG. 10B). These results strongly indicate the existence of stable complexes between RPTPe and Yes and between RPTPe and Fyn, both of which are consistent with either kinase being a substrate of RPTPe.

Expression of Yes or of Fyn does not rescue the altered morphology of RPTPe-deficient tumor cells: Results presented herein indicate that lack of RPTPe reduces activities of the Yes and Fyn kinases in EKO/Neu cells. Since lack of RPTPe is also the ultimate cause of the EKO/Neu cell phenotype, studies were conducted in efforts of uncovering if this phenotype could be reversed by increased expression of Yes or of Fyn as had been shown previously with Src [Gil-Henn and Elson, 2003 ibid]. To this end, EKO/Neu cells that had been infected with retroviral vectors expressing c-Yes or c-Fyn were used along with similar cells infected with a vector encoding c-Src or with an empty vector (positive and negative controls, respectively).

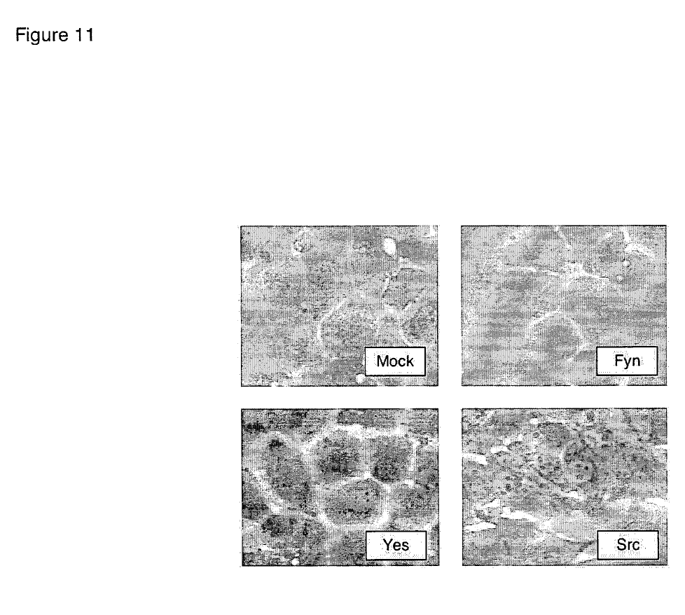

Cells expressing Src appeared smaller and grew in a more compact pattern than vector-infected cells. In general, cells expressing Src resembled Neu cells that express RPTPe; this indicates that morphological change could be induced and detected in the system used. In contrast, no significant changes in the morphology of cells expressing either Yes or Fyn was noted (FIG. 11). Expression of constitutively-active Y535F Yes induced very slight changes in morphology of the cells, while Y531F Fyn had no effect at all (not shown). Together these results indicate that reduced activities of Yes and of Fyn are not able to rescue the altered morphology of RPTPe-/−/Neu mammary tumor cells, and that despite their similarity to Src they perform distinct functions in these cells.

The above described results indicate that the non receptor-type form of PTPe, cyt-PTPe, can in principle also activate Yes and Fyn.

That lack of RPTPe would affect activities and phosphorylation of Yes and Fyn is in itself somewhat surprising, due to presence of several PTPs that can fulfill the roles of the absent RPTPe. The results provided herein suggest that RPTPe, RPTPa, and possibly other PTPs activate Yes and Fyn in Neu-induced mammary tumor cells, but that lack of RPTPe creates a significant deficit in the enzymatic activity required for optimal activation of Src-family kinases. Partial activity of Yes and Fyn in the absence of RPTPe and the ability of exogenous RPTPe or RPTPa to activate Src and to partially rescue the morphology of EKO/Neu cells [Gil-Henn and Elson 2003 ibid] agree with the above interpretation. In any case, the existence of cellular and molecular phenotypes in EKO/Neu cells indicate that absence of RPTPe is not compensated for by other PTPs in this cell system and suggests that similar substrate specificity among PTPs might not always translate into full functional redundancy.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents, patent applications and sequences identified by their accession numbers mentioned in this specification are herein incorporated in their entirety by reference into the specification, to the same extent as if each individual publication, patent, patent application or sequence identified by its accession number was specifically and individually indicated to be incorporated herein by reference. In addition, citation or identification of any reference in this application shall not be construed as an admission that such reference is available as prior art to the present invention.

What is claimed is:

1. A method of treating mammary tumors in a subject, the method comprising at least partially inhibiting receptor-type tyrosine phosphatase epsilon (RPTPe) activity or expression in mammary tumor tissue of the subject.

2. The method of claim 1, wherein said at least partially inhibiting is effected by introducing into said mammary tumor tissue an agent selected from the group consisting of:
(a) a molecule which binds RPTPe;
(b) an enzyme which cleaves RPTPe;
(c) an antisense polynucleotide capable of specifically hybridizing with an mRNA transcript encoding RPTPe;
(d) a ribozyme which specifically cleaves RPTPe transcripts;
(e) a non-functional analogue of at least a catalytic or binding portion of RPTPe;
(f) a molecule which prevents RPTPe activation or substrate binding;
(g) an siRNA molecule capable of inducing degradation of RPTPe transcripts; and
(h) a DNAzyme which specifically cleaves RPTPe transcripts or DNA.

3. The method of claim 2, wherein said antisense polynucleotide includes a sequence selected from the group consisting of SEQ ID NOs: 1-3.

4. The method of claim 2, wherein said non-functional analogue is capable of binding site of Src.

5. The method of claim 2, wherein said non-functional analogue is a substrate-trapping mutant of RPTPe.

6. The method of claim 1, wherein said at least partially inhibiting is accomplished by gene knockout.

7. The method of claim 2, wherein said introducing is effected via systemic administration of said agent.

8. The method of claim 1, wherein said subject is a human being.

9. A pharmaceutical composition for treating mammary tumors, the composition comprising, as an active ingredient, a therapeutically effective amount of an agent capable of at least partially inhibiting RPTPe activity or expression and a physiologically acceptable carrier and/or excipient.

10. The pharmaceutical composition of claim 9, wherein said agent capable of at least partially inhibiting RPTPe is selected from the group consisting of:
(a) a molecule which binds RPTPe;
(b) an enzyme which cleaves RPTPe;
(c) an antisense polynucleotide capable of specifically hybridizing with an mRNA transcript encoding RPTPe;
(d) a ribozyme which specifically cleaves RPTPE transcripts;
(e) a non-functional analogue of at least a catalytic or binding portion of RPTPe;
(f) a molecule which prevents RPTPe activation or substrate binding;
(g) an siRNA molecule capable of inducing degradation of RPTPe transcripts; and
(h) a DNAzyme which specifically cleaves RPTPe transcripts or DNA.

11. The pharmaceutical composition of claim 10, wherein said antisense polynucleotide includes a sequence selected from the group consisting of SEQ ID NOs: 1-3.

12. The pharmaceutical composition of claim 10, wherein said non-functional analogue is capable of binding a RPTPe binding site of Src.

13. The pharmaceutical composition of claim 10, wherein said non-functional analogue is a substrate-trapping mutant of RPTPe.

14. The pharmaceutical composition of claim 9, wherein said agent capable of at least partially inhibiting RPTPe is a phosphatase inhibitor.

15. An article of manufacture comprising packaging material and a pharmaceutical composition identified for treatment of mammary tumors being contained within said packaging material, said pharmaceutical composition including, as an active ingredient, an agent capable of at least partially inhibiting RPTPe activity or expression and a pharmaceutically acceptable carrier.

16. The article of manufacture of claim 15, wherein said agent is selected from the group consisting of:
(a) a molecule which binds RPTPe;
(b) an enzyme which cleaves RPTPe;
(c) an antisense polynucleotide capable of specifically hybridizing with an mRNA transcript encoding RPTPe;
(d) a ribozyme which specifically cleaves RPTPe transcripts;
(e) a non-functional analogue of at least a catalytic or binding portion of RPTPe;
(f) a molecule which prevents RPTPe activation or substrate binding;
(g) an siRNA molecule capable of inducing degradation of RPTPe transcripts; and
(h) a DNAzyme which specifically cleaves RPTPe transcripts or DNA.

17. The article of manufacture of claim 16, wherein said antisense polynucleotide includes a sequence selected from the group consisting of SEQ ID NOs 1-3.

18. The article of manufacture of claim 16, wherein said non-functional analogue is capable of binding a RPTPe binding site of Src.

19. The article of manufacture of claim 16, wherein said non-functional analogue is a substrate-trapping mutant of RPTPe.

20. A method of reducing morphologic transformation and proliferation rate in a cell, cell culture or tissue, the method comprising at least partially inhibiting RPTPe activity or expression in the cell, cell culture or tissue.

21. The method of claim 20, wherein said at least partially inhibiting is accomplished by genetic manipulation of the cell, cell culture or tissue.

22. The method of claim 21, wherein said genetic manipulation includes a knockout of RPTPe.

23. A method of identifying a drug candidate for treatment of mammary tumors comprising screening a plurality of molecules for a molecule capable of at least partially inhibiting RPTPe activity or expression, said molecule capable of inhibiting RPTPe activity or expression being the drug candidate.

24. The method of claim 23, wherein said screening is accomplished by measuring at least one parameter selected from the group consisting of RPTPe binding, specific binding to an RPTPe transcript, RPTPe cleavage, RPTPe activity and binding to an RPTPe binding site.

25. The method of claim 24, wherein said RPTPe binding site is a binding site on Src.

26. The method of claim 23, wherein said screening is effected by at least one method selected from the group consisting of an antibody based assay, an assay for competitive inhibition of RPTPe binding, an assay of inhibition of RPTPe activity, an assay of specific RPTPe binding, an assay of specific binding to at least a portion of an RPTPe transcript and an assay of RPTPe molecular weight.
